(19) United States
(12) Patent Application Publication
(10) Pub. No.: US 2013/0324892 A1
(43) Pub. Date: Dec. 5, 2013

(54) ULTRASONIC MEANS AND METHODS FOR DORSAL ROOT GANGLION NEUROMODULATION

(71) Applicant: BOSTON SCIENTIFIC NEUROMODULATION CORPORATION, Valencia, CA (US)

(72) Inventors: Changfang Zhu, Valencia, CA (US); Kerry Bradley, Glendale, CA (US)

(21) Appl. No.: 13/904,940

(22) Filed: May 29, 2013

Related U.S. Application Data
(60) Provisional application No. 61/652,840, filed on May 29, 2012.

(51) Int. Cl. A61N 7/00

(52) U.S. Cl. CPC A61N 7/00
USPC 601/2

(57) ABSTRACT

A method of treating a patient with an ailment, comprises delivering ultrasound energy to a dorsal root ganglia (DRG), thereby modulating the DRG to treat the ailment.

### RELATED APPLICATION DATA

The present application claims the benefit under 35 U.S.C. §119 to U.S. provisional patent application Ser. No. 61/652,840, filed May 29, 2012. The foregoing application is hereby incorporated by reference into the present application in its entirety.

### FIELD OF THE INVENTION

The present invention relates to tissue modulation systems, and more particularly, to a system and method for therapeutically modulating nerve fibers.

### BACKGROUND OF THE INVENTION

Among many techniques attempted for neurostimulation (e.g., electrical, chemical, mechanical, thermal, magnetic, optical, and so forth), electrical stimulation is the standard and most common technique. Implantable electrical stimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) techniques, which directly stimulate the spinal cord tissue of the patient, have long been accepted as a therapeutic modality for the treatment of chronic pain syndromes, and the application of spinal cord stimulation has begun to expand to additional applications, such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems such as the Freehand system by NeuroControl (Cleveland, Ohio) have been applied to restore some functionality to paralyzed extremities in spinal cord injury patients. Occipital Nerve Stimulation (ONS), in which leads are implanted in the tissue over the occipital nerves, has shown promise as a treatment for various headaches, including migraine headaches, cluster headaches, and cervicogenic headaches. In recent investigations, Peripheral Stimulation (PS), which includes Peripheral Nerve Field Stimulation (PNFS) techniques that stimulate nerve tissue directly at the symptomatic site of the disease or disorder (e.g., at the source of pain), and Peripheral Nerve Stimulation (PNS) techniques that directly stimulate bundles of peripheral nerves that may not necessarily be at the symptomatic site of the disease or disorder, has demonstrated efficacy in the treatment of chronic pain syndromes and incontinence, and a number of additional applications are currently under investigation. Vagal Nerve Stimulation (VNS), which directly stimulate the Vagal Nerve, has been shown to treat heart failure, obesity, asthma, diabetes, and constipation.

Each of these implantable stimulation systems typically includes at least one stimulation lead implanted at the desired stimulation site and neurostimulator (e.g., an implantable pulse generator (IPG)) implanted remotely from the stimulation site, but coupled either directly to the electrode lead(s) or indirectly to the stimulation lead(s) via a lead extension. Thus, electrical pulses can be delivered from the neurostimulator to the stimulation lead(s) to stimulate or activate a volume of neural tissue. In particular, electrical energy conveyed between at least one cathodic electrode and at least one anodic electrode creates an electrical field, which when strong enough, depolarizes (or “stimulates”) the neurons beyond a threshold level, thereby inducing the firing of action potentials (APs) that propagate along the neural fibers. The stimulation regimen will typically be one that provides stimulation energy to all of the target tissue that must be stimulated in order to provide the therapeutic benefit, yet minimizes the volume of non-target tissue that is stimulated.

The stimulation system may further comprise a handheld remote control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon. If the IPG contains a rechargeable battery, the stimulation system may further comprise an external charger capable of transcutaneously recharging the IPG via inductive energy.

Recently, there has been an interest in stimulating dorsal root ganglia (DRG) for the treatment of chronic pain. The DRG is a nodule that contains cell bodies of neurons of afferent spinal nerves, and in particular, dorsal root (DR) nerve fibers. Afferent spinal nerves provide sensory information (such as touch, pain, heat/cold, and proprioceptive sensation) which is propagated by action potentials that travel along the nerve fibers. As shown in FIG. 1, a DRG 1 comprises cell bodies 2 (or somas) that include axon branches projecting to central and peripheral targets. In particular, each cell body 2 is typically connected to a stem neural axon 3 that is branched to a central neural axon 4 (i.e., a spinal nerve) that extends to the spinal cord, and a peripheral neural axon 5 that extends to a peripheral region of the human body. The positioning of the cell body 2 is somewhat midway between the central neural axon 4 and the peripheral neural axon 5, and thus, may be called “pseudounipolar.”

Traditionally, a cell soma provides metabolic support, but DRG soma are known to undergo subthreshold depolarization when neighbor soma are invaded with afferent spikes. This means that some degree of cross-talk between the cell bodies mayoccur in the DRG. In healthy DRG, these interactions tend to be causal, in that regular afferent activity will generate subthreshold oscillations and some spiking while the afferent signaling is present, but rarely when sensory neurons are quiet. In pathological states, such as those following nerve injury or trauma, it is believed that the DRG soma become hyperactive, such that they generate enhanced periodic subthreshold membrane oscillations, often independent of afferent activity. In the hyperactive state, the soma have increased metabolic needs, and these needs may lead to oxygen debt and reduced mitochrondrial performance with the sensory neurons. This, in turn, can lead to ectopic electrical spiking within the sensory neurons. The action potentials resulting from the ectopic electrical spiking then feed into the dorsal horn laminae and are believed to hypersensitize these neural structures. This hypersensitization may then lead to chronic pain.

There, thus, remains a need to provide techniques for treating chronic pain.

### SUMMARY OF THE INVENTION

In accordance with the present inventions, a method of treating a patient with an ailment (e.g., pain) is provided. The method comprises delivering (e.g., epidurally) ultrasound energy to a dorsal root ganglia (DRG), thereby modulating the DRG to treat the ailment. The ultrasound energy may have a frequency in the range of 20 KHz-2 MHz. The ultrasound energy may be delivered from at least one ultrasonic transducer implanted within the patient. The ultrasonic transducer may comprise an array of ultrasonic transducers. In one method, the frequency of the ultrasound energy is relatively low (e.g., in the range of 20 KHz-100 KHz), thereby heating the DRG. In another method, the frequency of the ultrasound energy is relatively high (e.g., greater than 1 MHz), thereby increasing blood flow to the DRG. An optional method further comprises delivering a pharmacological agent to the DRG, thereby further modulating the DRG to treat the ailment. In this case, the delivery of the ultrasound energy may induce sonicphoresis for the pharmacological agent in the DRG. Another optional method further comprises delivering ultrasound energy to a central neural axon and/or peripheral neural axon extending from the DRG, thereby modulating the central neural axon and/or peripheral neural axon to treat the ailment.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a prior art plan view of a dorsal root ganglion (DRG) and surrounding neural structures;

FIG. 2 is a plan view of one embodiment of a neuromodulation system arranged in accordance with the present inventions;

FIG. 3-3A are plan views of a fully implantable modulator (FIM) and catheter used in the neuromodulation stimulation system of FIG. 2;

FIG. 4 is front view of a remote control (RC) used in the neuromodulation system of FIG. 2;

FIG. 5 is a block diagram of the internal components of the RC of FIG. 4;

FIG. 6 is a plan view of the neuromodulation system of FIG. 2 in use within the spinal column a patient for treating chronic pain; and

FIG. 7 is a cross-sectional view of the use of the catheter of FIG. 3 for modulating a dorsal root ganglion (DRG).

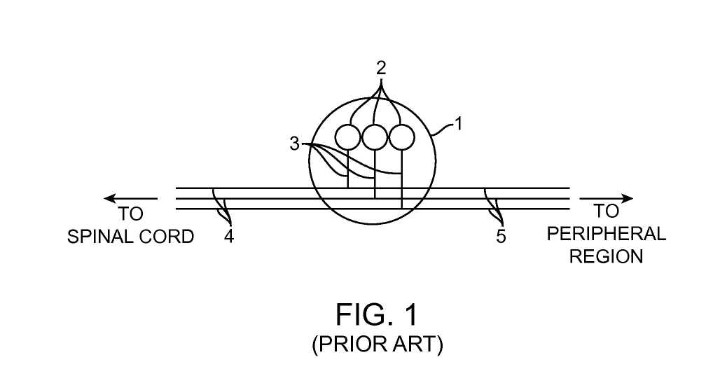

### DETAILED DESCRIPTION OF THE EMBODIMENTS

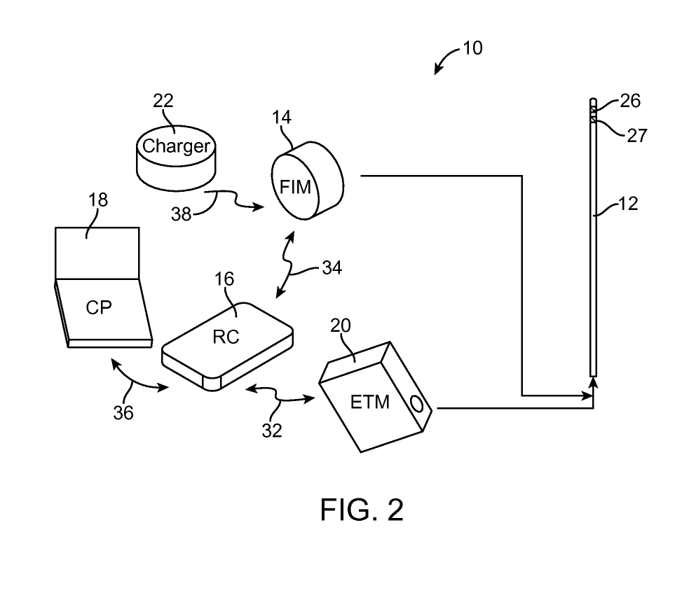

Turning first to FIG. 2, an exemplary neuromodulation system 10 is used to ultrasonically and optionally pharmacologically modulate the dorsal root ganglion (DRG) and surrounding neural structures. The system 10 generally includes an ultrasonic/drug delivery catheter 12, a fully implantable modulator (FIM) 14, an external control device in the form of a remote controller (RC) 16, a clinician's programmer (CP) 18, an external trial modulator (ETM) 20, and an external charger 22.

The catheter 12 includes one or more ultrasonic transducers 26, which emit ultrasound energy when excited by electrical energy. Ultrasound energy is a mechanical wave in frequencies beyond human hearing. The catheter 12 also includes one or more drug delivery ports 27 from which a pharmacological agent may be delivered. The FIM 14 is physically connected to the catheter 12. As will be described in further detail below, the FIM 14 delivers appropriate electrical energy to the ultrasonic transducer(s) 26 in accordance with a set of ultrasound neuromodulation parameters, as well as a pharmacological agent to the drug delivery port 27 in accordance with drug delivery parameters. Although only one catheter 12 is shown, multiple ultrasonic/drug delivery catheters may be connected to the FIM 14.

The ETM 20 may also be physically connected to the catheter 12. The ETM 20, which has similar components as that of the FIM 14, also delivers the ultrasound energy to the ultrasonic transducer(s) 26 and a pharmacological agent to the drug delivery port 27. The major difference between the ETM 20 and the FIM 14 is that the ETM 20 is a non-implantable device that is used on a trial basis after the catheter 12 has been implanted and prior to implantation of the FIM 14, to test the responsiveness of the neuromodulation that is to be provided. Thus, any functions described herein with respect to the FIM 14 can likewise be performed with respect to the ETM 20.

The RC 16 may be used to telemetrically control the ETM 20 via a bi-directional RF communications link 32. Once the FIM 14 and neuromodulation leads 12 are implanted, the RC 16 may be used to telemetrically control the FIM 14 via a bi-directional RF communications link 34. Such control allows the FIM 14 to be turned on or off and to be programmed with different neuromodulation parameter sets. The FIM 14 may also be operated to modify the programmed neuromodulation parameters to actively control the characteristics of the ultrasound energy/pharmacological agent output by the FIM 14 to the ultrasonic transducer(s) 26 and drug delivery port 27.

The CP 18 provides clinician detailed neuromodulation parameters for programming the FIM 14 and ETM 20 in the operating room and in follow-up sessions. The CP 18 may perform this function by indirectly communicating with the FIM 14 or ETM 20, through the RC 16, via an IR communications link 36. Alternatively, the CP 18 may directly communicate with the FIM 14 or ETM 20 via an RF communications link (not shown). The clinician detailed neuromodulation parameters provided by the CP 18 are also used to program the RC 16, so that the neuromodulation parameters can be subsequently modified by operation of the RC 16 in a stand-alone mode (i.e., without the assistance of the CP 18). The external charger 22 is a portable device used to transcutaneously charge the FIM 14 via an inductive link 38. Once the FIM 14 has been programmed, and its power source has been charged by the external charger 22 or otherwise replenished, the FIM 14 may function as programmed without the RC 16 or CP 18 being present.

For purposes of brevity, the details of the CP 18, ETM 20, and external charger 22 will not be described herein. Details of exemplary embodiments of these devices are disclosed in U.S. Pat. No. 6,895,280, which is expressly incorporated herein by reference.

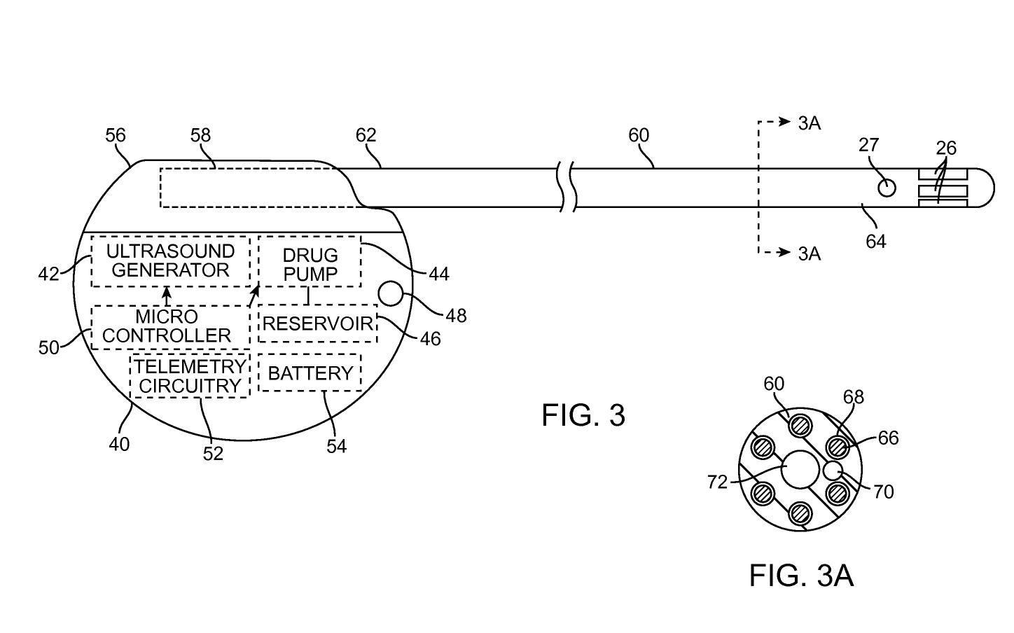

Referring to FIG. 3, the FIM 14 comprises an outer case 40 for housing the electronic and other components (described in further detail below). The outer case 40 is composed of an electrically conductive, biocompatible material, such as titanium, and forms a hermetically sealed compartment wherein the internal electronics are protected from the body tissue and fluids. The FIM 14 comprises an ultrasound generator 42 configured for generating electrical pulses for exciting the ultrasonic transducer(s) 26 on the catheter 12, and a drug pump 44 configured for conveying a pharmacological agent (e.g., an anesthetic, such as lidocaine, bupivacain, ropivacaine, and chloroprocaine, and/or an opioid, such as morphine, fentanyl, sufentanil, or pethidine) stored in a reservoir 46 to the drug delivery port 27 on the catheter 12. The ultrasound generator 42 is capable of generating electrical pulses in the ultrasound range (20 Khz-2 MHz), which electrical pulses will ultimately be emitted as ultrasound energy by the ultrasonic transducer(s) 26 on the catheter 12. The ultrasound generator 42 may generate the electrical pulses as a pulse train, or in continuous fashion, or in a burst fashion in the range of a few microseconds to several minutes. The drug pump 44 is capable of delivering the pharmacological agent at an adjustable bolus rate in the range of 0.1-24 mL/per day.

The FIM 14 may optionally include an external sealed port access 48 for refilling the reservoir 46 with a pharmacological agent using a hypodermic needle (not shown). Alternatively, if a drug pump is not available, the hypodermic needle may be used to directly supply a pharmacological agent via to the port access 48 to the drug delivery port 27 on the catheter 12.

The FIM 14 further comprises a microcontroller 50 carrying out a program function in accordance with a suitable program stored in memory (not shown). Thus, the microcontroller 50 generates the necessary control and status signals, which allow the microcontroller 50 to control the operation of the FIM 14 in accordance with a selected operating program and neuromodulation parameters. In accordance with neuromodulation parameters stored within the memory, the microcontroller 50 may control the amplitude and frequency of electrical pulses generated by the ultrasound generator 42, as well as the drug delivery rate of the drug pump 44. The memory also store a schedule for periodically delivering the therapeutic ultrasound energy and/or pharmacological agent.

The FIM 14 further comprises telemetry circuitry 52 (including antenna) configured for receiving programming data (e.g., the operating program and/or neuromodulation parameters) from the RC 16 in an appropriate modulated carrier signal, and demodulating the carrier signal to recover the programming data, which programming data is then stored within the memory. The telemetry circuitry 52 also provides status data to the RC 16.

The FIM 14 further comprises a rechargeable power source 54 for providing the operating power to the FIM 14. The rechargeable power source 54 may, e.g., comprise a lithium-ion or lithium-ion polymer battery. The rechargeable power source 54 is recharged using rectified AC power (or DC power converted from AC power through other means, e.g., efficient AC-to-DC converter circuits, also known as “inverter circuits”) received by an AC receiving coil (not shown). To recharge the power source 54, the external charger 22 (shown in FIG. 2), which generates the AC magnetic field, is placed against, or otherwise adjacent, to the patient's skin over the implanted FIM 14. The AC magnetic field emitted by the external charger 22 induces AC currents in the AC receiving coil. Charging circuitry (not shown) rectifies the AC current to produce DC current, which is used to charge the power source 54.

It should be noted that rather than having a fully contained FIM, the system 10 may alternatively utilize an implantable receiver-modulator (not shown) connected to the catheter 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-modulator. The implanted receiver-modulator receives the signal and delivers the therapy in accordance with the control signals.

The FIM 14 further comprises a connector 56 to which the catheter 12 mates in a manner that couples the ultrasonic transducer(s) 26 and drug delivery port 27 to the internal components within the outer case 40. To this end, the connector 56 includes a port 58 (shown in phantom) for receiving the proximal end of the catheter 12.

The catheter 12 includes an elongated catheter body 60 having a proximal end 62 and a distal end 64. The catheter body 60 may, e.g., have a diameter within the range of 0.03 inches to 0.07 inches and a length within the range of 10 cm to 90 cm. The catheter body 60 may be composed of a suitable electrically insulative material, such as, a polymer (e.g., polyurethane or silicone), and may be extruded from as a unibody construction.

The catheter 12 includes the ultrasonic transducer(s), which in the illustrated embodiment, takes the form of a transducer array 26 circumferentially disposed around the distal end 64 of the body 60 for emitting ultrasound energy in the radial direction. As such, the delivery of the ultrasound energy to the DRG is not influenced by the rotation of the catheter 12 relative to the DRG. That is, active ultrasound transducer may be directed towards the DRG, but if catheter rotation occurs, this misdirection of the ultrasound energy may be corrected by selecting a different ultrasound transducer that is directed towards the DRG after the catheter 12 has rotated. Alternatively, the multiple radially oriented ultrasonic transducers may be driven at different intensities (sonic pressure) levels and/or frequencies to implement a form of “ultrasound steering” where the peak sonic pressure level is administered at a point between the ultrasonic transducers.

The catheter 12 further comprises the drug delivery port 27 from which the pharmacological agent is delivered. The drug delivery port 27 is located on one side of the catheter body 60. Alternatively, multiple drug delivery ports 27 may be arranged around the circumference of the catheter body 60 in much the same as the radially-oriented ultrasonic transducers 26.

The catheter 12 further comprises a connector (not shown) mounted to the proximal end 62 of the catheter body 60, which mates with the connector 56 of the FIM 14 for respectively coupling the ultrasound generator 42 and drug pump 44 to the ultrasonic transducer(s) 26 and drug delivery port 27 mounted to the distal end 64 of the catheter body 60.

As shown in FIG. 3A, the catheter 12 further comprises a plurality of electrical conductors 66 housed within individual lumens 68 extending within the catheter body 60 between the connector (not shown) carried by the proximal end 62 of the catheter body 60 and the transducer array 26 carried by the distal end 64 of the catheter body 60 using suitable means, such as welding. In this manner, the transducers 26 may be individually and independently excited by the FIM 14. The catheter 12 further comprises a drug lumen 70 extending through the catheter body 60 between the connector (not shown) carried by the proximal end 62 of the catheter body 60 and the drug delivery port 27 carried by the distal end 64 of the catheter body 60. The catheter 12 further comprises a central lumen 72 that may be used to accept an insertion stylet (not shown) to facilitate implantation of the catheter 12. The connector includes electrical terminals (not shown) hardwired to the respective conductors 66 and capable of mating with corresponding electrical terminals (not shown) on the connector 56 of the FIM 14 (shown in FIG. 2). Thus, electrical energy can be conveyed from the FIM 14 to the catheter connector, along the conductors 66 to the transducer array 26. The connector includes a fluid coupler (not shown) affixed to the drug lumen 70 and capable of mating with a corresponding fluid coupler on the connector 56 of the FIM 14 (shown in FIG. 2). Thus, a pharmacological agent can be conveyed from the FIM 14 to the catheter connector, along the drug lumen 70 to the drug delivery port 27.

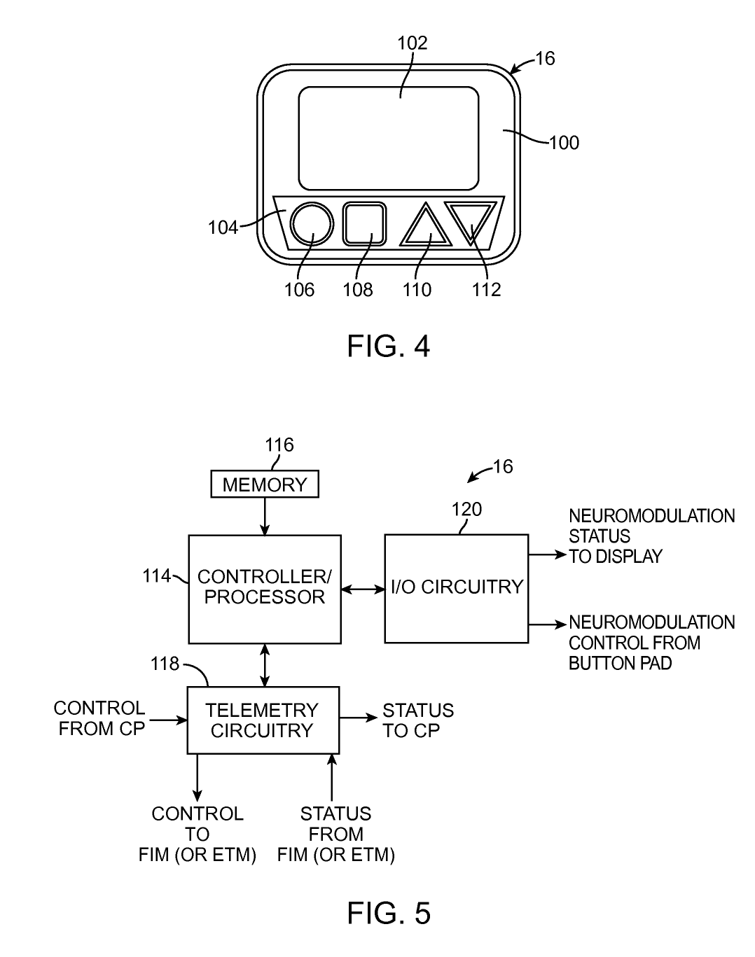

Referring now to FIG. 4, one exemplary embodiment of an RC 16 will now be described. As previously discussed, the RC 16 is capable of communicating with the FIM 14 or CP 18. The RC 16 comprises a casing 100, which houses internal componentry (including a printed circuit board (PCB)), and a lighted display screen 102 and button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. In an optional embodiment, the display screen 102 has touchscreen capabilities. The button pad 104 includes a multitude of buttons 106, 108, 110, and 112, which allow the FIM 14 to be turned ON and OFF, provide for the adjustment or setting of neuromodulation parameters within the FIM 14, and provide for selection between screens.

In the illustrated embodiment, the button 106 serves as an ON/OFF button that can be actuated to turn the FIM 140N and OFF. The button 108 serves as a select button that allows the RC 106 to switch between screen displays and/or parameters. The buttons 110 and 112 serve as up/down buttons that can be actuated to increase or decrease any of neuromodulation parameters of the electrical energy generated by the FIM 14, including the amplitude and frequency, which will ultimately be transformed by the ultrasonic transducer array 26 into ultrasound energy having intensity (sonic pressure) levels and frequencies dictated by the amplitude and frequency of the electrical energy generated by the FIM 14.

The selection button 158 can be actuated to place the RC 16 in an “Ultrasound Modulation Adjustment Mode,” during which any of the ultrasound neuromodulation parameters, including the amplitude and frequency, can be selected and adjusted via the up/down buttons 160, 162, or an “Drug Delivery Adjustment Mode,” during which any of the pharmacological neuromodulation parameters, including the flow rate, can be selected and adjusted via the up/down buttons 160, 162. Alternatively, dedicated up/down buttons can be provided for each neuromodulation parameter. Rather than using up/down buttons, any other type of actuator, such as a dial, slider bar, or keypad, can be used to increment or decrement the neuromodulation parameters. Thus, the RC 16 can be used to program the FIM 14 with the desired intensity and frequency of the therapeutic ultrasound energy and the desired bolus rate of the drug delivery in a calibration procedure. The RC 16 may also store information about the programming/calibration session, as well as uploaded information from the FIM 14, which can log diagnostics of use over time.

Referring to FIG. 5, the internal components of an exemplary RC 16 will now be described. The RC 16 generally includes a controller/processor 114 (e.g., a microcontroller), memory 116 that stores an operating program for execution by the controller/processor 114, and telemetry circuitry 118 for transmitting control data (including neuromodulation parameters and requests to provide status information) to the FIM 14 and receiving status information from the FIM 14 via link 34 (or link 32) (shown in FIG. 2), as well as receiving the control data from the CP 18 and transmitting the status data to the CP 18 via link 36 (shown in FIG. 2). The RC 16 further includes input/output circuitry 120 for receiving stimulation control signals from the button pad 104 and transmitting status information to the display screen 102 (shown in FIG. 5). Further details of the functionality and internal componentry of the RC 16 are disclosed in U.S. Pat. No. 6,895,280, which has previously been incorporated herein by reference.

Having described the structure and function of the neuromodulation system 10, a technique for using the neuromodulation system 10 to treat patients having pain, which may be chronic, will now be described. In this method, ultrasound neuromodulation energy is delivered to a dorsal root ganglia (DRG), thereby modulating the DRG. Modulation of the DRG and surrounding neural structures modulates the sensory information, such as pain, touch, heat, and proprioceptive sensation traveling through the DRG. The effect on the DRG and surrounding neural structures will depend on the frequency of the ultrasound neuromodulation energy. If the frequency of the ultrasound neuromodulation energy is relatively low (e.g., 20 KHz-100 KHz) with a relatively higher sonic pressure level, the pressure and vibration may induce changes in tensions on the neuronal membrane of the DRG cell bodies. The voltage gated ion channels may be sensitive to such mechanical tension changes, thereby changing the membrane conduction and thus excitability of the cell bodies. If the frequency of the ultrasound neuromodulation energy is relatively high (e.g., greater than 1 MHz) with a relatively lower sonic pressure level, the blood flow to the DRG may be increased, thereby providing more oxygen and increased mitochondrial performance of the DRG cell bodies. A pharmacological agent may also be delivered to the DRG as an adjunct to the delivery of the ultrasound energy. Advantageously, the delivery of the ultrasound energy to the DRG may improve the sonicphoresis of the pharmacological agent in the DRG.

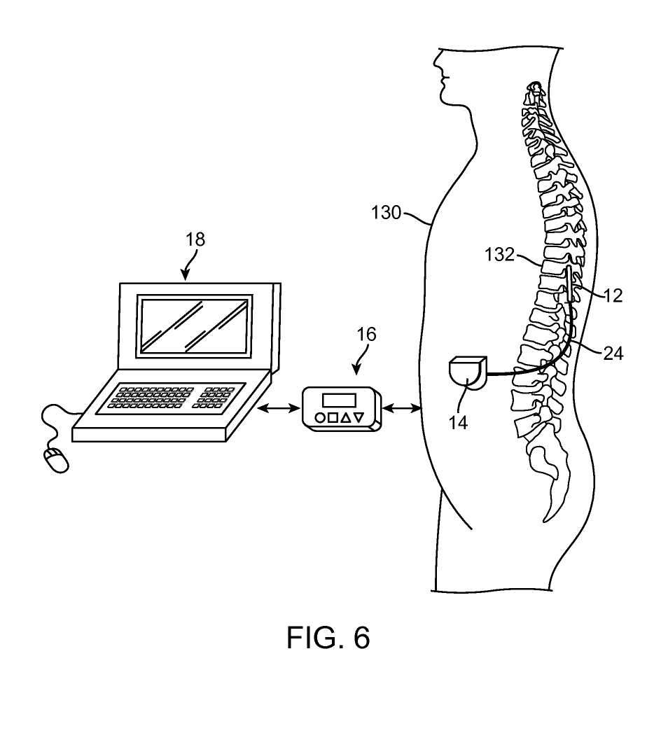

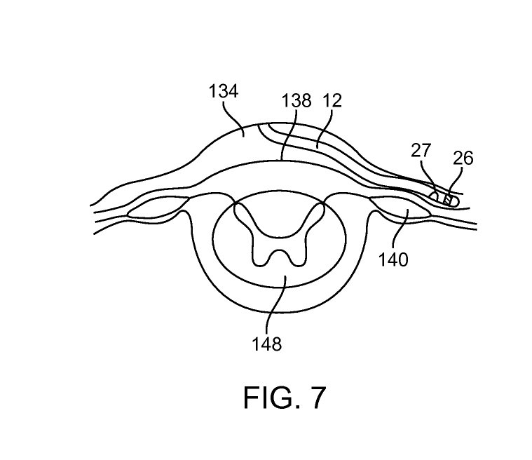

In one method for treating chronic pain, the DRG and optionally the surrounding neural structures may be modulated by implanting the catheter 12 within the spinal column 132 of a patient 130, as shown in FIG. 6. As shown in FIG. 7, the preferred placement of the catheter 12 is in the epidural space 134 of the patient 130. The catheter 12 may be located in the foramen 136 that extends from the epidural space 134 over the dura 138 covering the DRG 140. In this manner, ultrasound neuromodulation energy and pharmacological can be conveniently delivered to the DRG 140 and surrounding neural structures. The catheter 12 can conventionally be introduced, with the aid of fluoroscopy, into the epidural space 134 above the spinal cord 148 through a Touhy-like needle, which passes through the skin, between the desired vertebrae, and into the epidural space 134 above the dura 138. In many cases, a stylet, such as a metallic wire, is inserted into a lumen running through the center of the catheter 12 to aid in insertion of the lead through the needle and into the epidural space 134. The catheter 12 may then be introduced into the foramen 136 from the epidural space 134. The stylet gives the lead rigidity during positioning, and once the catheter 12 is positioned, the stylet can be removed after which the lead becomes flaccid.

After proper placement of the catheter 12 at the target area of the spinal column 132, the catheter 12 is anchored in place to prevent movement of the catheter 12. The proximal end of the catheter 12 exiting the spinal column 132 is passed through one or more tunnels (not shown) subcutaneously formed along the torso of the patient 130 to a subcutaneous pocket (typically made in the patient's abdominal or buttock area) where the FIM 14 is implanted. The FIM 14 may, of course, also be implanted in other locations of the patient's body. A subcutaneous tunnel can be formed using a tunneling tool over which a tunneling straw may be threaded. The tunneling tool can be removed, the catheter 12 threaded through the tunneling straw, and then the tunneling straw removed from the tunnel while maintaining the catheter 12 in place within the tunnel.

The catheter 12 is then connected directly to the FIM 14 by inserting the proximal end of the catheter 12 within the connector port 58 located on the connector 56 of the FIM 14. The FIM 14 can then be operated to generate the electrical energy for exciting the ultrasonic transducers 26 and/or delivering the pharmacological agent through the drug delivery port 27. As there shown, the CP 18 communicates with the FIM 14 via the RC 16, thereby providing a means to control and reprogram the FIM 14.

The ultrasound neuromodulation energy and/or pharmacological agent may also be delivered from the catheter 12 to neural structures surrounding the DRG 140, including central neural axon and peripheral neural axon extending from the DRG 140. In this case, additional ultrasound transducers and/or drug delivery ports may be provided along the length of the catheter 12, such that the ultrasound neuromodulation energy and/or pharmacological agent may be concurrently delivered to both the DRG and the central neural axon and/or peripheral neural axon. The delivery of the ultrasound neuromodulation energy and/or pharmacological agent to either the central neural axon or peripheral neural axon may depend on the source of the pain. For example, if the source of pain resides only in the DRG 140, the ultrasound energy and/or pharmacological agent can be delivered to the both the DRG 140 and the central neural axon. If the source of pain resides only in the peripheral neural axon, the ultrasound energy and/or pharmacological agent can be delivered to the both the DRG 140 and the peripheral neural axon. If the source of pain resides in both the DRG 140 and the peripheral neural axon, the ultrasound energy and/or pharmacological agent can be delivered to all three of the DRG 140, the central neural axon, and the peripheral neural axon.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method of treating a patient with an ailment, comprising:
delivering ultrasound energy to a dorsal root ganglia (DRG), thereby modulating the DRG to treat the ailment.

2. The method of claim 1, wherein the ultrasound energy has a frequency in the range of 20 KHz-2 MHz.

3. The method of claim 1, wherein the ultrasound energy has a frequency in the range of 20 KHz-100 KHz, thereby heating the DRG.

4. The method of claim 1, wherein the ultrasound energy has a frequency greater than 1 MHz, thereby increasing blood flow to the DRG.

5. The method of claim 1, further comprising delivering a pharmacological agent to the DRG, thereby further modulating the DRG to treat the ailment.

6. The method of claim 5, wherein the delivery of the ultrasound energy induces sonicphoresis for the pharmacological agent in the DRG.

7. The method of claim 1, wherein the ultrasound energy is epidurally delivered to the DRG.

8. The method of claim 1, wherein the ultrasound energy is delivered from at least one ultrasonic transducer implanted within the patient.

9. The method of claim 8, wherein the at least one ultrasonic transducer comprises an array of ultrasonic transducers.

10. The method of claim 1, further comprising delivering ultrasound energy to a central neural axon extending from the DRG, thereby modulating the central neural axon to treat the ailment.

11. The method of claim 1, further comprising delivering the ultrasound energy to a peripheral neural axon extending from the DRG, thereby modulating the peripheral neural axon to treat the ailment.

12. The method of claim 1, wherein the ailment is pain.
